(19) United States
(12) Patent Application Publication
(10) Pub. No.: US 2025/0339704 A1
(43) Pub. Date: Nov. 6, 2025

(54) Method and Apparatus for Accurately Positioning a Transcranial Magnetic Stimulation (TMS) Coil

(71) Applicant: Christian Hirschbeck, Munich (DE)

(72) Inventor: Christian Hirschbeck, Munich (DE)

(21) Appl. No.: 19/198,007

(22) Filed: May 3, 2025

Related U.S. Application Data
(60) Provisional application No. 63/642,693, filed on May 4, 2024.

(51) Int. Cl. A61N 2/00; A61N 2/02

(52) U.S. Cl. CPC A61N 2/006; A61N 2/02

(57) ABSTRACT

The present invention relates to an apparatus for accurately positioning a transcranial magnetic stimulation (TMS) coil over a target position on a patient's head. The apparatus includes a TMS coil with an integrated real-time display device that shows the position of the coil relative to the patient's head. A processor for calculating the coils spatial coordinates using a three-dimensional tracking method and transmitting the information to the display. A synchronization mechanism providing real-time updates of display as the TMS coil moves thus, providing immediate feedback to the operator. This enables precise alignment of the coil with the target position. The invention offers advantages by improving treatment accuracy, reducing errors and providing user-friendly, real-time feedback for precise coil positioning. The scope of the invention extends to clinical settings, brain research and therapeutic applications where it can enhance treatment consistency and support expanded uses of TMS in medical practice and research.

### CROSS REFERENCE TO RELATED APPLICATION

This disclosure claims the benefit of the priority of U.S. Provisional Patent Application No. 63/642,693, entitled “METHOD AND APPARATUS FOR ACCURATELY POSITIONING A TRANSCRANIAL MAGNETIC STIMULATION (TMS) COIL” and filed on May 4, 2024. The above-identified application is incorporated herein by reference in its entirety.

### TECHNICAL FIELD

The present invention relates to the field of transcranial magnetic stimulation (TMS), and more particularly to a system and method for precise and repeatable positioning of a TMS coil over a targeted region of a patient's head during the treatment of neurological or psychiatric disorder.

### BACKGROUND

The background description includes information that may be useful in understanding the present invention. It is not an admission that any of the information provided herein is prior art or relevant to the presently claimed invention, or that any publication specifically or implicitly referenced is prior art.

Transcranial Magnetic Stimulation (TMS) is a non-invasive neuromodulation technique that utilizes rapidly changing magnetic fields to induce electric currents in specific regions of the brain. Since development, TMS has gained considerable attention for its ability to modulate neural activity without the need for surgery or implanted devices. It has found widespread application in both clinical and research settings with particular promise in the treatment of neurological and psychiatric conditions such as major depressive disorder (MDD), obsessive-compulsive disorder (OCD) and chronic pain syndromes. In research domains, TMS serves as a valuable tool for probing functional connectivity and neuroplasticity in the brain.

Traditionally, TMS therapy involves the use of an electromagnetic coil that is placed near the scalp over the region of interest. When current passes through the coil, it generates a magnetic field that penetrates the skull and induces localized electrical currents in the underlying cortical tissue. The efficacy and reproducibility of TMS therapy are highly dependent on the precise positioning of the coil with respect to the intended cortical target. Even small deviations in coil placement can lead to variations in stimulation efficacy, decreased therapeutic benefit or unintended stimulation of adjacent brain regions.

Historically, two main approaches have been employed for coil positioning: manual and computerized navigation. Manual positioning methods are based on anatomical landmarks, visual approximation and surface measurements using tools such as measuring tapes or template caps. Clinicians may mark the scalp based on standard coordinates (e.g., the 10-20 EEG system) to estimate the location of the desired target. Although this approach is relatively simple and cost-effective, it is inherently prone to human error, variability between sessions and inter-operator inconsistency. Reproducing coil placement with high accuracy across multiple treatment sessions remains a significant challenge with manual techniques.

To address the limitations of manual methods, neuronavigation systems were introduced. The systems use real-time imaging data, often including MRI scans combined with motion tracking technologies to precisely guide the placement of the TMS coil over the patient's head. While neuronavigation systems significantly enhance the accuracy and consistency of coil positioning, they are associated with considerable drawbacks. These include high acquisition and maintenance costs, increased procedural complexity and extended setup time. Furthermore, such systems typically require trained personnel and a dedicated space making them less feasible for routine use in busy clinical environments or smaller practices.

Despite the known benefits of accurate coil positioning, the field has lacked a practical solution that combines the precision of neuronavigation with the simplicity and affordability of manual methods. The absence of such a solution has created a critical barrier to the wider adoption of TMS therapy in everyday clinical settings. As TMS becomes more accepted as a frontline treatment for mental health and neurological conditions, the demand for a robust, user-friendly and cost-effective targeting system continues to grow.

The challenges led to the pressing need for a novel system that provides accurate, repeatable and intuitive positioning of the TMS coil without the complexity or expense of traditional neuronavigation systems.

### SUMMARY

The present invention relates to a method and apparatus for accurately and repeatably positioning a transcranial magnetic stimulation (TMS) coil over a target position on a patient's head during treatment for neurological or psychiatric disorders. The invention provides a TMS coil with a housing integrated with or attached to a real-time display device that is connected to one or more processors. The processors are configured to calculate the position of the coil relative to the patient's head and communicate this data to the display in real time. Synchronization mechanisms ensure that the display reflects live movements of the coil, allowing the operator to visually align the coil with the target area with precision. The scope of the invention covers both the method and apparatus for real-time feedback-guided positioning of a TMS coil applicable to a wide range of clinical and therapeutic settings. The invention overcomes the limitations of manual coil placement and the complexity of existing computerized systems by offering a more practical, accessible and cost-effective solution. The invention includes various advantages i.e. advanced positioning accuracy, simplified clinical operation, increased treatment consistency and improved patient outcomes. By enabling precise and stable coil alignment, the apparatus supports more effective stimulation therapy, reduces operator error and facilitates repeatable treatment sessions with minimal setup time.

In an embodiment of the present invention, the invention discloses an apparatus for accurately positioning of a transcranial magnetic stimulation (TMS) coil over a target position on a patient's head. The apparatus comprises a TMS coil having a housing. The TMS coil is configured to position on a patient's head. In addition, a real-time display device is integrated into the housing and operable to display a position of the TMS coil relative to the patient's head. The apparatus includes at least one processor configured to calculate the position of the TMS coil relative to the patient's head and communicate the position of the coil to the real-time display device. Additionally, a computer-readable medium stores instruction that are executable by the processor to control the display of coil position in real time and a synchronization mechanism is configured to synchronize the display of the real-time display with the movement of the TMS coil. Further, the positioning of the TMS coil is carried out by continuously calculating the spatial coordinates of the coil relative to the patient's head using a three-dimensional tracking method and displaying the coordinates on the real-time display device to enable visual alignment of the coil with the target position on the patient's head.

In one of the embodiments of the present invention, the real-time display device is shielded against electromagnetic radiation to prevent interference with the operation of the display device during the positioning of the TMS coil.

In one of the embodiments of the present invention, the display device is configured to virtually display anatomical structures inside the patient's head.

In one of the embodiments of the present invention, the apparatus further comprises locking means configured to lock the TMS coil in a fixed position once the target position is achieved.

In one of the embodiments of the present invention, the locking means includes a mechanical or electromagnetic locking mechanism that secures the TMS coil in a fixed place.

In one of the embodiments of the present invention, an alert system is configured to notify the operator when the position of the TMS coil deviates from the fixed position during the treatment session.

In one of the embodiments of the present invention, the synchronization mechanism includes a feedback loop that ensures continuous real-time alignment of the TMS coil with the target position on the patient's head.

In one of the embodiments of the present invention, the real-time display device may attach to the housing of the TMS coil.

In another embodiment of the present invention, the invention discloses a method for accurately positioning a transcranial magnetic stimulation (TMS) coil over a target position on a patient's head. The method comprising selecting a TMS coil having a housing and integrating or attaching a real-time display device to the housing. In addition, the method includes attaching of the display device to at least one processor to calculate and transmit the position of the TMS coil relative to the patient's head to the display device. Further, synchronization of the display of the real-time display device with movement of the TMS coil is used to provide real-time visual feedback to an operator. In addition, the method adjusts the position of the TMS coil using the visual feedback to align the coil precisely with the target location on the patient's head.

In one of the embodiments of the present invention, the real-time display device is shielded against electromagnetic radiation generated by the TMS coil to prevent interference with the display and a computer unit.

In one of the embodiments of the present invention, the at least one processor is configured to execute feedback algorithms that continuously calculate and adjust the displayed position of the TMS coil relative to the patient's head.

In one of the embodiments of the present invention, the method further comprises virtually locking of TMS coil in place after achieving the desired treatment position.

In one of the embodiments of the present invention, the position of the TMS coil is displayed on the real-time display device using three-dimensional tracking method.

In one of the embodiments of the present invention, an alert system detects unintended movement of the TMS coil during treatment and generates a user alert in response to the detected changes.

For further clarification of the features and other embodiments of the invention, a more particular description is provided that will further explain the features and advantage of the invention with the illustration or the drawings. As will be appreciated, other embodiments of the present invention are possible utilizing, alone or in combination, one or more of the features set forth above or described in detail below.

### BRIEF DESCRIPTION OF THE DRAWINGS

A full understanding of the invention can be gained from the following description of the preferred embodiments when read in conjunction with the accompanying drawings in which:

FIG. 1 illustrates a block diagram showcasing overall components of an apparatus configured for accurately positioning a transcranial magnetic stimulation (TMS) coil over a target position on a patient's head, in according to embodiments of the present invention disclosed herein.

FIG. 2 illustrates a perspective view of an apparatus configured for positioning a transcranial magnetic stimulation (TMS) coil over the patient's head, in according to embodiments of the present invention disclosed herein.

FIG. 3 illustrates a bottom perspective view of a display mount, in according to embodiments of the present invention disclosed herein.

FIG. 4A illustrates a front perspective view of the apparatus for positioning a transcranial magnetic stimulation (TMS) coil over the patient's head, in according to embodiments of the present invention disclosed herein.

FIG. 4B illustrates an anterior perspective view of the apparatus for positioning a transcranial magnetic stimulation (TMS) coil over the patient's head, in according to embodiments of the present invention disclosed herein.

FIG. 5 illustrates a cross-sectional view of the apparatus, in according to embodiments of the present invention disclosed herein.

FIG. 6 illustrates an application of using the apparatus for positioning a transcranial magnetic stimulation (TMS) coil over the patient's head for treatment of neurological or psychiatric disorders, in according to embodiments of the present invention disclosed herein.

FIG. 7 illustrates a method for accurately positioning a transcranial magnetic stimulation (TMS) coil over a target position on a patient's head during treatment of a neurological or psychiatric disorder, in according to embodiments of the present invention disclosed herein.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION

References will now be made in detail to the presently preferred embodiments of the invention, examples of which are illustrated in the accompanying drawings. Throughout the following detailed description, the same reference numerals refer to the same elements in all figures.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and it is not intended to be limiting the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In the following description, reference will be made to the accompanying drawing, in which comparable functional elements are designated with like numerals. The aforementioned accompanying drawings show by way of illustration and not by the way of limitation, specific aspects and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in limited sense. It is noted that description herein is not intended as an extensive overview, and as such, concepts may be simplified in the interests of clarity and brevity. All documents mentioned in this application are hereby incorporated by reference in their entirety.

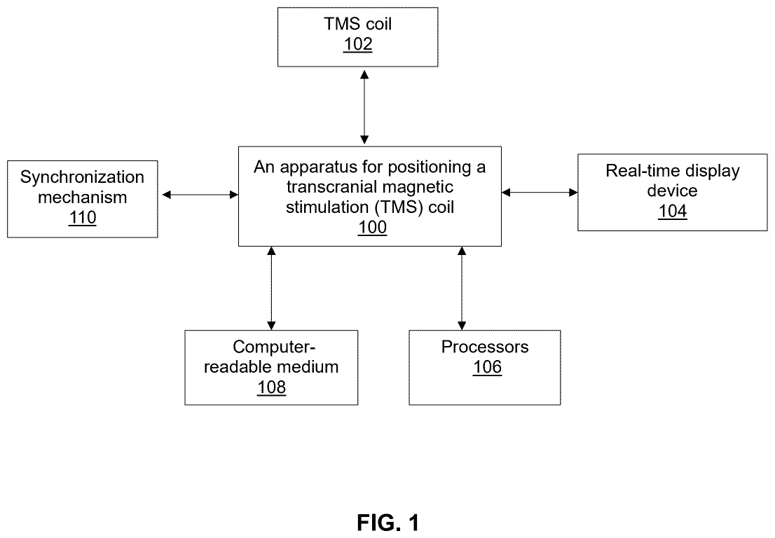

According to the embodiment of the present invention, an apparatus for accurately positioning a transcranial magnetic stimulation (TMS) coil over a target position on a patient's head is disclosed in FIG. 1. In the embodiment, the apparatus 100 includes a TMS coil 102 comprising a housing. The TMS coil 102 is composed of high-conductivity material i.e. copper. The TMS coil 102 is responsible for generating the magnetic fields that are directed at the patient's head. In the embodiment, a real-time display device 104 is integrated directly into or attached to the housing of the TMS coil 102. The real-time display device 104 is configured to show the position of the TMS coil 102 in relation to the patient's head. The real-time display device 104 is equipped with a user-friendly interface that allows the operator to observe and monitor the exact location of the coil as it is moved over the patient's head. The real-time display 104 provides immediate visual feedback helping the operator adjust the coil's position to achieve precise alignment with the target brain region. The real-time nature of the display 104 ensures that any necessary adjustments can be made immediately, improving the overall accuracy of the treatment. The real-time display device 104 can be shielded against electromagnetic radiation. The real-time display device 104 comprises a touch screen. Since the TMS coil 102 generates electromagnetic fields to produce the magnetic pulses necessary for treatment, these fields could interfere with the display's operation. The shielding ensures that the real-time display remains fully functional, unaffected by the electromagnetic interference generated by the TMS coil 102. In addition, the apparatus 100 also includes at least one processor 106 that are in communication with the real-time display device 104. The processor 106 is configured to calculate the precise position of the TMS coil 102 in three-dimensional space relative to the patient's head. The spatial coordinates for determining the position of TMS coil 102 is calculated using 3D tracking method. The tracking can be accomplished using technologies such as optical tracking, magnetic tracking or other sensor-based methods that monitor the position of the TMS coil 102 in three-dimensional space. This calculation is necessary for ensuring that the TMS coil 102 is positioned accurately over the desired target area on the patient's head. The positioning data is then transmitted to the real-time display device 104 which updates the display in real time. The processors 106 rely on a computer-readable medium 108 such as memory storage to store instructions that guide the movement of the TMS coil 102, ensuring its alignment with the target location on the head. In the embodiment, the apparatus 100 includes a synchronization mechanism 110 which ensures that the display of the TMS coil's position is synchronized with its movement. The synchronization is achieved through a feedback loop which continuously adjusts the visual representation of the coil's position as it is moved by the operator. The feedback loop allows for continuous real-time adjustments which is vital for keeping the coil in the correct position throughout the treatment session.

In one of the embodiments of the present invention, the apparatus 100 includes locking means that help secure the TMS coil in a fixed position once it has been properly aligned with the target brain region. The locking mechanism can either be mechanical or electromagnetic thus, providing a secure way to hold the coil in place during the treatment process. Once the coil is locked into position, it ensures that the magnetic field is consistently directed at the intended location without any unintended deviations.

In one of the embodiments of the present invention, the display is connected to a computer system that is responsible for calculating and communicating the position of the TMS coil in relation to the patient's head.

In one of the embodiments of the present invention, an alert system is incorporated into the apparatus 100 to notify the operator if the TMS coil 102 deviates from its fixed position during the treatment. This is particularly useful for maintaining consistent and accurate stimulation throughout the procedure. The alert system can be set to activate if the coil shifts beyond a certain threshold, ensuring that the operator can take corrective action quickly.

In one of the embodiments of the present invention, TMS is a noninvasive procedure that uses magnetic fields to stimulate nerve cells in the brain to improve symptoms of neurological or psychiatric disorders. Existing devices and methods for TM are known and described in the art (see, e.g., Lefaucheur, J-P. et al. Attorney Docket No. 10198-001PV1 8 “Evidence-based guidelines on the therapeutic use of repetitive transcranial magnetic stimulation (rTMS): An update (2014-2018).” Clin. Neurophysiol. 131 (2):474-528 (2020); Burke, M. J., Fried P. J., & Pascual-Leone A. “Transcranial magnetic stimulation: Neurophysiological and clinical applications.” Handb. Clin. Neurol. 163:73-92 (2019); Vucic S. et al. “Clinical diagnostic utility of transcranial magnetic stimulation in neurological disorders. Updated report of an IFCN committee.” Clin. Neurophysiol. 150:131-175 (2023); Lefaucheur J. P. “Transcranial magnetic stimulation.” Handb. Clin. Neurol. 160:559-580 (2019); Iglesias A. H. “Transcranial magnetic stimulation as treatment in multiple neurologic conditions.” Curr. Neurol. Neurosci. Rep. 20(1): 1(2020); Gogulski J. et al. “Personalized repetitive transcranial magnetic stimulation for depression.” Biol. Psychiatry Cogn. Neurosci. Neuroimaging. 8(4):351-360 (2023); and Jannati A. et al. “Assessing the mechanisms of brain plasticity by transcranial magnetic stimulation.” Neuropsychopharmacology. 48(1): 191-208 (2023); Richter K., Kellner S., & Licht C. “rTMS in mental health disorders.” Front. Netw. Physiol. 3:943223 (2023)).

In one of the embodiments of the present invention, the computer-usable or computer-readable medium include but not limited to an electronic, magnetic, optical, electromagnetic, infrared, or semiconductor system, apparatus, device, or propagation medium. The computer readable medium may include transitory and/or non-transitory embodiments. The computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD ROM), an optical storage device, a transmission medium such as those supporting the Internet or an intranet, or a magnetic storage device. The computer-usable or computer-readable medium could even be paper or another suitable medium upon which the program is printed, as the program can be electronically captured, via, for instance optical scanning of the paper or other medium, then compiled, interpreted, or otherwise processed in a suitable manner, if necessary, and then stored in a computer memory. Further, a computer usable or computer-readable medium may be any medium that can contain, store, communicate, propagate or transport the program for use by or in connection with the instruction execution system, apparatus, or device.

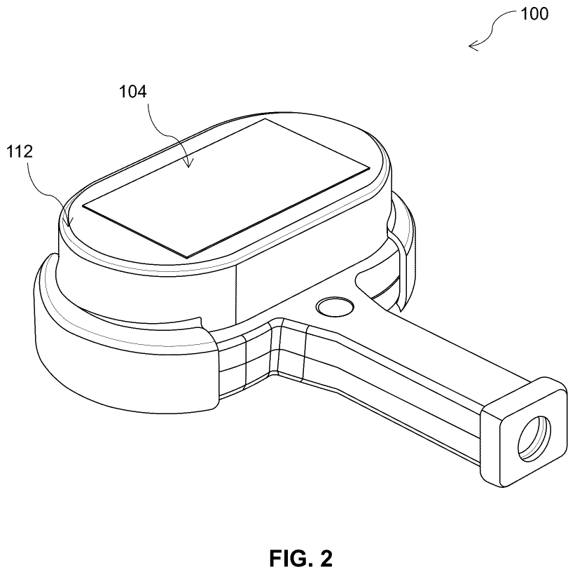

Referring to FIG. 2, an apparatus 100 for positioning a transcranial magnetic stimulation (TMS) coil is disclosed. The apparatus 100 includes a real-time display device 104 and a display mount 112 both configured to work in tandem to facilitate accurate and consistent positioning of the TMS coil during treatment. The real-time display device 104 is a compact, high-resolution visual interface that provides real-time feedback on the spatial relationship between the TMS coil and the patient's head. The real-time display device 104 receives continuous input from a connected processing unit that calculates the coil's exact position using 3D tracking data. This information is then displayed visually to guide the clinician in aligning the coil precisely over the target stimulation area. The real-time display device 104 is engineered to function reliably in close proximity to the electromagnetic field generated by the coil and is therefore equipped with electromagnetic shielding to prevent signal interference and maintain display clarity during operation. In addition, the display mount 112 is a structurally stable, adjustable support that secures the display device 104 either directly to the TMS coil housing or in a fixed position near it. The display mount 112 is made from lightweight, non-ferromagnetic materials. The display mount 112 is configured to withstand the magnetic environment without compromising safety or performance. It allows for tilt, rotation and height adjustments, ensuring ergonomic viewing angles for the operator regardless of the coil's orientation. The flexibility ensures that the real-time feedback is always easily visible during the positioning and treatment process. The display mount 112 also includes locking mechanisms to hold the display in a fixed position once adjusted, preventing unwanted movement during clinical use.

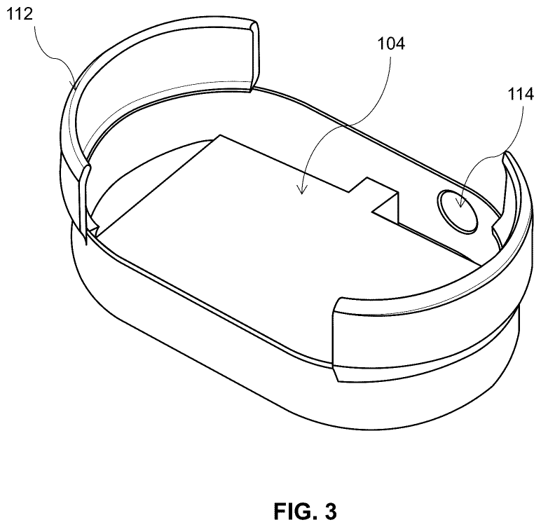

Referring to FIG. 3, bottom view of the display mount is disclosed. The apparatus comprises a display mount 112 securely connected to or integrated with the TMS coil housing configured to support a real-time display device 104 used for visualizing coil positioning relative to the patient's head. The display mount offers mechanical stability while allowing for adjustable viewing angles through tilt or swivel features. In addition, a set of cable holes 114 facilitate clean and secure passage of cables such as those for power, data or video signals between the real-time display device 104 and an external processing system (e.g., a computer or control unit). The cable holes 114 are strategically placed along the housing or mount structure to reduce clutter, prevent cable entanglement and protect connections from wear due to movement during coil handling. The routing design also helps shield sensitive cables from electromagnetic interference by allowing proper separation and integration with shielding materials where necessary. Further, a bottom side of the real-time display device 104 may house internal shielding layers to protect the display's electronic components from the strong electromagnetic fields generated by the TMS coil. The design ensures that all functional aspects of the display i.e. connectivity, secure mounting are compactly integrated and accessible thus, contributing to the overall reliability and usability of the system.

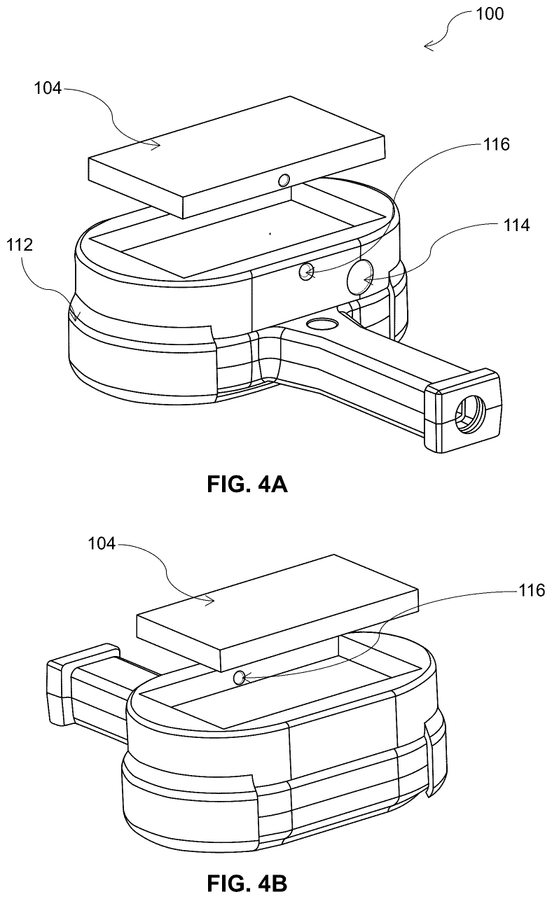

Referring to FIG. 4A-FIG. 4B, various different sides of an apparatus for positioning a transcranial magnetic stimulation (TMS) coil is disclosed. According to FIG. 4A, a front perspective view of the apparatus 100 is disclosed. The apparatus 100 includes a display mount 112 which serves as the structural support for the real-time display device 104. The display mount 112 is affixed either directly to the TMS coil housing or to an articulated arm that extends from the main coil assembly. The real-time display device 104 is then fixed securely onto the display mount 112 using a display fixation mechanism 116 such as precision screws, brackets or locking interfaces. This ensures that the real-time display device 104 remains in a consistent position during coil movement and operation. The fixation mechanism 116 not only prevents vibration or displacement but also maintains alignment between the visual interface and the coil's spatial orientation. In addition, the cables connecting the display to the external computer or processing unit are routed through cable holes 114 which are integrated into the display mount 112 or coil housing. The holes 114 are positioned to follow a natural path from the display 104 to the external system, minimizing interference, reducing cable movement and preventing accidental disconnection. The cable paths may also include additional shielding or insulation to protect signal integrity. In the embodiment, the FIG. 4B discloses the anterior perspective view of the apparatus. The apparatus showcases real-time display device 104 which is prominently visible and oriented toward the operator. The view highlights the position of the real-time display device 104 on the TMS coil housing or mount, the fixation mechanism 116 securing the display and providing a clear view of how the operator would interact with the system during coil positioning and treatment.

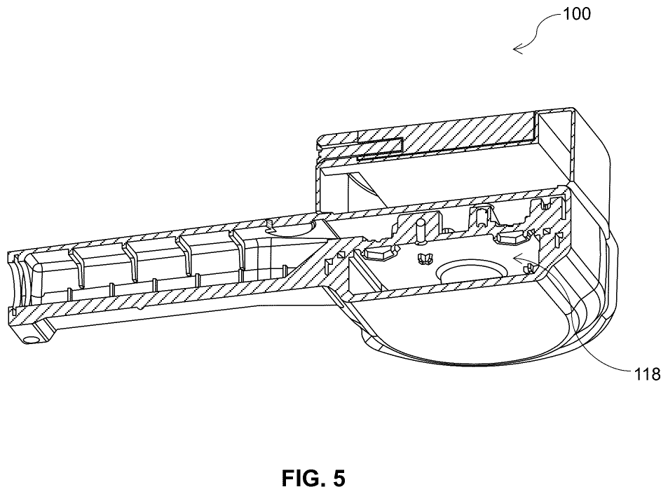

Referring to FIG. 5, a cross-sectional view of the apparatus is disclosed. The apparatus 100 comprises a TMS coil having a housing 118 that serves as the primary structural enclosure for the coil and associated components. The housing 118 is configured to be both durable and electromagnetically compatible and is made from non-ferromagnetic materials to prevent interference with the magnetic fields generated during stimulation. The housing 118 provides a protective shell for internal elements such as the real-time display device, tracking sensors, electromagnetic shielding and cable routing infrastructure. The housing is shaped to provide coil positioning and enabling precise alignment of coil over the patient's head.

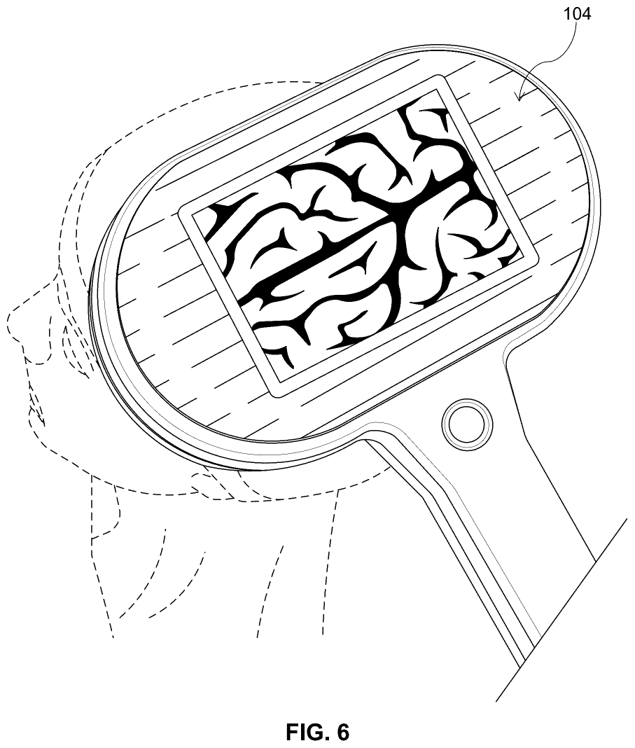

Referring to FIG. 6, an application of the apparatus is disclosed. In the embodiment, the apparatus comprises the TMS coil integrated with a real-time display device 104 which plays a critical role in assisting the clinician with accurate coil placement. The display device 104 is actively monitoring and displaying the anatomical structure of the patient's brain by providing a real-time visual overlay that guides positioning relative to the targeted stimulation area. The display device 104 achieves this by interfacing with an external computer system that processes pre-acquired brain imaging data (e.g., MRI or CT scans) registered to the patient's head. A 3D tracking model detects the position and orientation of the coil in real time allowing the software to map the coil's location relative to the internal brain structures. The mapping is then visualized on the display as an interactive, real-time image that effectively allows the clinician to see through the brain. The real-time display device 104 shows internal brain regions such as cortical targets as if they are visible beneath the coil. As the operator adjusts the coil, the display updates instantly showing changes in alignment and providing visual cues to help reach the exact location of interest. Once aligned, the apparatus can confirm correct positioning and alert the user if the coil moves out of place during treatment. The real-time visualization enhances targeting accuracy, reducing setup time and ensuring consistent treatment delivery by clearly showing the spatial relationship between the coil and underlying anatomical brain structures on the real-time display device 104.

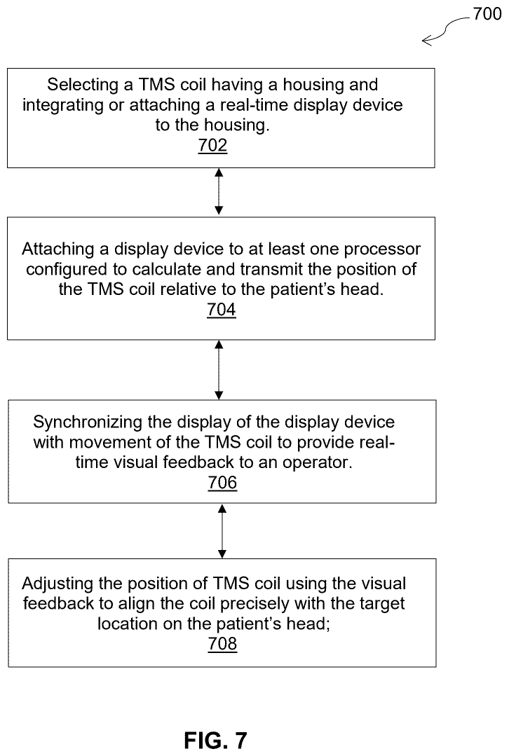

Referring to FIG. 7, a method for accurately and repeatably positioning of a transcranial magnetic stimulation (TMS) coil over a specific target location on a patient's head during treatment for neurological or psychiatric disorders is disclosed. In the embodiment, according to step 702, the method 700 begins with the selection of a TMS coil that incorporates or supports the integration of a real-time display device into its housing. In the embodiment, the display device is seamlessly embedded within the coil housing. In another embodiment, the display may be securely attached externally to the housing. The display device plays a pivotal role in providing real-time visual feedback directly to the operator during coil positioning. The display is specifically engineered to resist the electromagnetic interference generated by the TMS coil, ensuring that image fidelity and system operation are maintained during high-intensity magnetic pulse delivery. In addition, according to step 704, the display device is electronically linked to one or more processors via a communication network. The communications network may be a cable network operating in the radio-frequency domain and/or the Internet Protocol (IP) domain. The communications network may also include a distributed computing network such as the Internet (sometimes alternatively known as the “World Wide Web”), an intranet, a local-area network (LAN) and/or a wide-area network (WAN). The communications network may further include coaxial cables, copper wires, fiber optic lines or hybrid-coaxial lines. The communications network further includes wireless portions utilizing any portion of the electromagnetic spectrum and any signaling standard (such as the IEEE 802 family of standards, GSM/CDMA/TDMA or any cellular standard, and/or the ISM band). The invention may be applied to any wireless/wireline communications network, regardless of physical componentry, physical configuration, or communications standard(s). In some aspects, wireless communication interfaces may include but are not limited to an Intranet connection, Internet, Personal Area Networks (PA Ns) for the exchange of data over short distances, e.g., using short-wavelength radio transmissions in the industrial, scientific, and medical (ISM) band ISM band from 2400-2480 MHz) from fixed and mobile devices (e.g., 15 Bluetooth® technology), wireless fidelity (Wi-Fi), Wi-Max, IEEE 802.1 1 technology, radio frequency (RF), Infrared Data Association (IrDA) compatible protocols, Local Area Networks (LANs), Wide Area Networks (WANs), Shared Wireless Access Protocol (SWAP), Zigbee, Near-Field Communication (NFC), LiFi, 5G, any combinations thereof and other types of wireless networking protocols. The processors are equipped with software stored on computer-readable medium comprising executable instructions that perform several critical operations. The processors calculate the real-time position and orientation of the TMS coil relative to the patient's head using three-dimensional (3D) spatial tracking technology. This may involve the use of optical markers, inertial measurement units, electromagnetic tracking systems, or a combination of these to capture both translational and rotational movements of the coil. The tracking system can register the coil's movement in all six degrees of freedom (x, y, z axes and roll, pitch, yaw), providing highly accurate spatial data. These measurements may be referenced to predefined anatomical landmarks on the patient's scalp or brain-based targets identified via imaging (e.g., MRI or fMRI) depending on the clinical application. In the embodiment, in step 706, once the positional data is generated, it is transmitted from the processor to the display device. A synchronization mechanism is then employed to ensure that the movement of the TMS coil is immediately reflected on the real-time display. Synchronization refers to the closed-loop communication and real-time updating between the coil's physical motion and the corresponding on-screen visuals. As the operator manipulates the TMS coil moving it closer, rotating it or tilting it, the positional sensors detect the changes instantly and the processor computes the updated coordinates. The updates are then conveyed to the display with sub-second latency, maintaining a seamless and uninterrupted flow of information. The display renders the data visually using cues such as directional arrows, distance markers, color-coded accuracy indicators, or alignment crosshairs. The visual elements enable the precision during placement. The synchronized loop allows for immediate feedback and facilitates accurate coil maneuvering even in complex or high-throughput clinical settings. In the embodiment, the step 708 discloses coil alignment process using the real-time visual feedback provided on the display. The adjustment phase involves interpreting the feedback to manually reposition the coil until it is correctly aligned with the pre-defined target location on the patient's head. The system may display indicators comparing the current and ideal coil positions, angular misalignments and real-time distance values to help the operator make both coarse and fine-tuned adjustments. For instance, if the coil is too far off-axis, the screen may show a warning or shift its visual cues to direct the operator to rotate or move the device accordingly. This continuous loop of feedback and manual adjustment ensures that the TMS coil is not only positioned at the correct anatomical site but also oriented at the precise angle needed to deliver the magnetic field effectively to the underlying neural tissue. The capability is especially important for protocols targeting deep or functionally specific brain regions where millimeter-level accuracy can affect treatment outcomes.

In one of the embodiments of the present invention, the real-time display device is shielded against electromagnetic radiation generated by the TMS coil to prevent interference with the display and a computer unit.

In one of the embodiments of the present invention, the at least one processor is configured to execute feedback algorithms that continuously calculate and adjust the displayed position of the TMS coil relative to the patient's head.

In one of the embodiments of the present invention, the method further comprises virtually locking of TMS coil in place after achieving the desired treatment position.

In one of the embodiments of the present invention, an alert system detects unintended movement of the TMS coil during treatment and generates a user alert in response to the detected changes.

In one of the embodiments of the present invention, the technology of the present invention significantly improves the precision and reliability of TMS coil placement and revolutionizes TMS procedures by making them more accessible, efficient and effective in clinical practice. In particular, an advantage of the present invention over the state of the art relates to simplified and improved accuracy; the positioning of the TMS coil is greatly simplified, making the present invention more accessible for clinical use while increasing precision. In addition, the invention provides various advantages i.e. the technology can be readily used in clinical practice, thereby improving the practicality and efficiency of TMS procedures. The precise positioning of the TMS coil increases the efficacy and safety of TMS treatments which ultimately has a positive impact on patient outcomes.

In one of the embodiments of the present invention, the program code used for carrying out operations of the invention may be written in an object oriented programming language such as Java, Smalltalk, C++ or similar language. The program code for carrying out operations of the invention may also be written in conventional procedural programming languages such as the C programming language or similar programming languages. The program code may be executed by a processor, (application specific integrated circuit ASIC) or other component that executes the program code. The program code may be simply referred to as a software application that is stored in memory (such as the computer readable medium discussed above). The program code may cause the processor (processor-controlled device) to produce a graphical user interface (GUI) that is visually produced on a display device. The program code, however, may operate in any processor-controlled device such as a computer, server, personal digital assistant, phone, television, or any processor controlled device utilizing the processor and/or a digital signal processor. The program code may locally and/or remotely execute. The program code for example may be entirely or partially stored, accessed and downloaded in local memory of the processor-controlled device. A user's computer for example may entirely execute the program code or only partly execute the program code. Further, the program code may be a stand-alone software package that is at least partly executed on the user's computer or partly executed on a remote computer or entirely on a remote computer or server.

It should be understood that the examples provided herein are intended only for purposes of illustration and any number of other implementations is also contemplated. Additionally, the referenced examples (including the described rules and/or other techniques) can be combined in any number of ways.

Although an overview of the inventive subject matter has been described with reference to specific example implementations, various modifications and changes can be made to those implementations without departing from the broader scopes of implementation of the present disclosure. Such implementation of the inventive subject matter can be referred to herein, individually or collectively, by the term “invention” merely for convenience without intending to voluntarily limit the scope of this application to any single disclosure or inventive concept if more than one is, in fact is disclosed.

The implementations illustrated herein are described in sufficient detail to enable those skilled in the art to practice the teachings disclosed. Other implementations can be used and derived therefrom, such that structural substitutions and changes can be made without departing from the scope of this disclosure. The Detailed Description, therefore, is not to be taken in a limiting sense, and the scope of various implementations is defined only by the appended claims, along with the full range of equivalents to which such claims are entitled.

As used herein, the term “or” can be construed in either an inclusive or exclusive sense. Moreover, plural instances can be provided for resources or structures described herein as a single instance. These and other variations, modifications, additions, and improvements fall within a scope of implementations of the present disclosure as represented by the appended claims. The specification and drawings are, accordingly, to be regarded in an illustrative rather than a restrictive sense.

What is claimed is:

1. An apparatus for accurately positioning a transcranial magnetic stimulation (TMS) coil over a target position on a patient's head, the apparatus comprising:
a TMS coil having a housing, the TMS coil configured to position on a patient's head;
a real-time display device integrated into the housing and operable to display a position of the TMS coil relative to the patient's head;
at least one processor configured to calculate the position of the TMS coil relative to the patient's head and communicate the position of the coil to the real-time display device;
a computer-readable medium storing instructions executable by the processor to control the display of coil position in real time;
a synchronization mechanism configured to synchronize the display of the real-time display with the movement of the TMS coil;
wherein the positioning of the TMS coil is carried out by continuously calculating spatial coordinates of the coil relative to the patient's head using a three-dimensional tracking method and displaying the coordinates on the real-time display device to enable visual alignment of the coil with the target position on the patient's head.

2. The apparatus as claimed in claim 1, wherein the real-time display device is shielded against electromagnetic radiation to prevent interference with the operation of the display device during the positioning of the TMS coil.

3. The apparatus as claimed in claim 1, wherein apparatus further comprises locking means configured to lock the TMS coil in a fixed position once the target position is achieved.

4. The apparatus as claimed in claim 3, wherein the locking means includes a mechanical or electromagnetic locking mechanism that secures the TMS coil in a fixed place.

5. The apparatus as claimed in claim 1, wherein an alert system is configured to notify the operator when the position of the TMS coil deviates from the fixed position during the treatment session.

6. The apparatus as claimed in claim 1, wherein the synchronization mechanism includes a feedback loop that ensures continuous real-time alignment of the TMS coil with the target position on the patient's head.

7. The apparatus as claimed in claim 1, wherein the real-time display device may attach to the housing of the TMS coil.

8. A method for accurately positioning a transcranial magnetic stimulation (TMS) coil over a target position on a patient's head, the method comprising:
selecting a TMS coil having a housing and integrating or attaching a real-time display device to the housing;
attaching the display device to at least one processor to calculate and transmit the position of the TMS coil relative to the patient's head to the display device;
synchronizing the display of the real-time display device with movement of the TMS coil to provide real-time visual feedback to an operator;
adjusting the position of the TMS coil using the visual feedback to align the coil precisely with the target location on the patient's head.

9. A method as claimed in claim 8, wherein the real-time display device is shielded against electromagnetic radiation generated by the TMS coil to prevent interference with the display and a computer unit.

10. A method as claimed in claim 8, wherein the at least one processor is configured to execute feedback algorithms that continuously calculate and adjust the displayed position of the TMS coil relative to the patient's head.

11. A method as claimed in claim 8, wherein the method further comprising virtually locking of TMS coil in place after achieving the desired treatment position.

12. A method as claimed in claim 8, wherein the position of the TMS coil is displayed on the real-time display device using three-dimensional tracking method.

13. A method as claimed in claim 8, wherein an alert system detects unintended movement of the TMS coil during treatment and generates a user alert in response to the detected changes.
